SEQUENCE LISTING (39 sequences)

SEQ ID NO: 1 - DNA, 151 bp, artificial sequence
  unsure 19-32, 51-64, 83-96, 103-116, 147-151: ligated string of sequence tags example; n is a,t,g or c
tctagacagtctggagagnnnnnnnnnnnnnntctagacagtctggagagnnnnnnnnnnnnnntctagacagtctggagagnnnnnnnnnnnnnntctagannnnnnnnnnnnnnctctccagactgtctagacagtctggagagnnnnn

SEQ ID NO: 2 - DNA, 18 bp, artificial sequence
  PCR Primer
ccgcttttctggattcat

SEQ ID NO: 3 - DNA, 21 bp, artificial sequence
  MAGE Universal Adapter
tctagaggactgcgtgggcga

SEQ ID NO: 4 - DNA, 25 bp, artificial sequence
  unsure 24,25: MAGE Universal Adapter; n is a,t,g or c
cctcgcccacgcagtcctctagann

SEQ ID NO: 5 - DNA, 19 bp, artificial sequence
  MAGE PCR PRimer
cctcgcccacgcagtcctc

SEQ ID NO: 6 - DNA, 18 bp, artificial sequence
  MAGE PCR Primer
cggctgggtgtggcggac

SEQ ID NO: 7 - DNA, 16 bp, Mus musclus
ggcgacacgcgcacct

SEQ ID NO: 8 - DNA, 16 bp, Mus musculus
agggaggagatgtagt

SEQ ID NO: 9 - DNA, 16 bp, Mus musculus
actacatctcctccct

SEQ ID NO: 10 - DNA, 16 bp, Mus musculus
gggttgtcttcactct

SEQ ID NO: 11 - DNA, 16 bp, Mus musculus
cccaggatgtatagct

SEQ ID NO: 12 - DNA, 16 bp, Mus musculus
agtctggaaacctgtc

SEQ ID NO: 13 - DNA, 16 bp, Mus musculus
actacatctcctacct

SEQ ID NO: 14 - DNA, 16 bp, Mus musculus
aggtaagtgctgctgc

SEQ ID NO: 15 - DNA, 16 bp, Mus musculus
aggtaagtacaagctg

SEQ ID NO: 16 - DNA, 16 bp, Mus musculus
acctcagttgattcct

SEQ ID NO: 17 - DNA, 16 bp, Mus musculus
agctttgaattcatga

SEQ ID NO: 18 - DNA, 16 bp, Mus musculus
tttctcagggtagcct

SEQ ID NO: 19 - DNA, 16 bp, Mus musculus
tccgctcaatgtacct

SEQ ID NO: 20 - DNA, 16 bp, Mus musculus
agggttggactcaagg

SEQ ID NO: 21 - DNA, 16 bp, Mus musculus
actacatctcctccgt

SEQ ID NO: 22 - DNA, 16 bp, Mus musculus
cacgggggcggagcct

SEQ ID NO: 23 - DNA, 16 bp, Mus musculus
aggtaaccctgtgctg

SEQ ID NO: 24 - DNA, 16 bp, Mus musculus
aggtagactacttgtg

SEQ ID NO: 25 - DNA, 16 bp, Mus musculus
cgccatcgctctagct

SEQ ID NO: 26 - DNA, 16 bp, Mus musculus
aggctcatcagctgac

SEQ ID NO: 27 - DNA, 16 bp, Mus musculus
aggtgtggatccagag

SEQ ID NO: 28 - DNA, 16 bp, Mus musculus
aggtaggtctcgatct

SEQ ID NO: 29 - DNA, 16 bp, Mus musculus
agaatacgatacccag

SEQ ID NO: 30 - DNA, 16 bp, Mus musculus
agggcatttctattac

SEQ ID NO: 31 - DNA, 16 bp, Mus musculus
agctaatcaccaaagc

SEQ ID NO: 32 - DNA, 16 bp, Mus musculus
aggatccactgtttac

SEQ ID NO: 33 - DNA, 16 bp, Mus musculus
aggtcctgcctcagca

SEQ ID NO: 34 - DNA, 16 bp, Mus musculus
aggtcctgcctcagca

SEQ ID NO: 35 - DNA, 16 bp, Mus musculus
aggactgattgtggtg

SEQ ID NO: 36 - DNA, 16 bp, Mus musculus
agataagtttgttctg

SEQ ID NO: 37 - DNA, 16 bp, Mus musculus
aggtccagtagggacc

SEQ ID NO: 38 - DNA, 16 bp, Mus musculus
aggtatgatgacaggt

SEQ ID NO: 39 - DNA, 16 bp, Mus musculus
aggtgtacgaatgcga

(19) United States
(12) Patent Application Publication
(10) Pub. No.: US 2005/0153302 A1
(43) Pub. Date: Jul. 14, 2005

(54) Method for comprehensive identification of cell lineage specific genes

(76) Inventors: Steven C. Pruitt, Williamsville, NY (US); Alexander Maslov, Williamsville, NY (US)

Correspondence Address: HODGSON RUSS LLP, ONE M & T PLAZA, SUITE 2000, BUFFALO, NY, 14203-2391, US

(21) Appl. No.: 10/759,334

(22) Filed: Jan. 16, 2004

Related U.S. Application Data
(60) Provisional application No. 60/440,510, filed on Jan. 16, 2003.

(51) Int. Cl. C12Q 1/68; C12P 19/34; C12N 15/85

(52) U.S. Cl. 435/6; 435/455; 435/91.2

(57) ABSTRACT

This invention provides a method for identification and characterization of genes expressed during differentiation of cells. Cell lineage specific genes are identified in embryonic stem cells lines by unique vectors constructed to permit rapid characterization of expressed genes.

This application claims priority to U.S. Provisional application No. 60/440,510, filed on Jan. 16, 2003, the disclosure of which is incorporated herein by reference.

### FIELD OF THE INVENTION

The present invention relates generally to the field of identification of expressed genes and more particularly to method for characterization of cell lineage specific genes.

### DISCUSSION OF RELATED ART

Embryonic and somatic stem cells are considered to offer potential therapy for a large spectrum of diseases. Parkinson's, cardiomyopathies, and diabetes are a small subset of the potential diseases that could benefit from the effective application of these cells. However, one of the major impediments to the effective use of embryonic stem cells to treat a broad range of diseases is the lack of sufficient knowledge of the mechanisms leading to the differentiation of the required cell lineages.

A cell's lineage defines its relationship to the multipotent precursor that gave rise to it. The molecular mechanisms controlling the decision to differentiate towards one of two or more alternative lineages are of great interest for understanding the basic biology of embryonic development as well as homeostasis within somatic tissues. Understanding these mechanisms is also important for the practical application of stem cell therapy.

The use of cell-type specific gene expression has historically been a key molecular method for defining cell types and cell lineage relationships. For example, comparison of genes expressed in embryonic stem (ES) cells with those expressed in neural stem cells (NSCs) and hematopoietic stem cells (HSCs) suggests a closer relationship between ES and NSCs than HSCs since there are more genes expressed in common between ES cells and NSCs. More recent application of global approaches to surveying gene expression now make it possible to move beyond cell type identification to molecular phenotyping based on the expressed complement of genes. This phenotype can imply function and, to a first approximation, cell type is defined at the molecular level by the genes expressed.

The ability of global approaches for surveying gene expression to give insights into function is illustrated by a recent study employing microarray technology to provide a comprehensive look at those genes that are expressed within mouse embryonic stem cells and compare these to genes expressed in neural and hematopoietic stem cells (Ramalho-Santos et al., 2002, Ivanova et al., 2002). The results from these studies provide a profile of genes common to the three stem cell compartments and these serve as clues to the functions that may be required to maintain cells in an undifferentiated state. Expression profiles from these cells were compared to those of the lateral ventricle of the brain and the main population of the bone marrow and genes showing elevated expression in one or more stem cell populations were identified. These studies revealed 216 or 283 genes (Ramalho-Santos, 2002 and Ivanova et al., 2002, respectively) that are elevated in their expression in all three stem cell compartments and imply a core set of signaling pathways that may contribute to maintaining the properties of stem cells. Additionally, the observation that there are fewer differences between ES cells and NSCs may imply that these cell types are more closely related to each other than to HSCs.

Similar molecular profiles for other multipotent somatic stem cell compartments and differentiated cell types could reveal much about the functions and relationships between these different cell types. Large-scale efforts characterizing genes expressed within tissues, often as a function of an additional parameter (e.g. age, disease state), have been made by a number of laboratories. A public repository of many of these results is available through the NCBI's Gene Expression Omnibus (GEO; http://www.ncbi.nlm.nih.gov/entrez/guery.fcgi?db=eo). GEO includes data from large-scale characterizations using Serial Analysis of Gene Expression (SAGE) and microarray detection platforms. While these data are useful for identifying specific genes within a tissue that responds to an experimental variable, the complex cell mixtures within most tissues limit the value of such studies for providing a profile of the genes expressed within any specific cell type.

Genome wide phenotyping of gene expression within specific cell types can be accomplished if a means of isolating the relevant cell type is available. In the example cited above, effective methods exist for either growing the cells (ES cells, NSCs), or in the case of HSCs isolating relatively pure populations (from an unlimited starting cell population, bone marrow, using FACS). There are, however, a number of caveats and limitations to applying similar microarray based approaches for defining the genes expressed during the differentiation of these cells to specific cell lineages. For in vitro differentiation model systems, two means to address this issue are: 1) to optimize the fraction of cells that differentiate towards the target lineage and/or 2) to physically isolate or select for the desired lineage. In practice, the first of these methods is generally employed through the inclusion of growth factors or ligands, often empirically identified, which can shift the ratio of cells differentiating towards a given lineage. While helpful, pure populations of cells directed towards only a single target lineage are rarely obtained in practice. For many cell lineages that are induced based on cell-cell interactions during the differentiation process, directing differentiation towards a single lineage may not be possible without a-priori knowledge of all of the relevant signals, if at all.

Physical isolation of target lineages is feasible and presents an efficient method for their recovery at high purity. In the case of the hematopoietic system, cell surface markers allowing identification of most cell compartments are available and such approaches are already possible in principle. A potentially powerful alternative for other cell lineages, where cell surface markers are generally not known or available, is the use of FACS sorted cells to recover cells that are marked by EGFP expression within a specific cell lineage. This approach has been applied to define the molecular phenotype of Drosophila germline cells by linking EGFP expression to the vasa gene (Sano et al., 2002), to define oligodendrocytic lineages derived from mouse ES cells using an Olig 2-GFP knock-in (Xian et al., 2003).

While the above approaches are useful for comparative analysis of expressed genes at selected times, many genes, often those of greatest interest, are expressed only transiently during the establishment of a cell lineage and will not be reflected in the expression profile from differentiated cells. To address this issue, in one approach, the fate of cells that express a known marker gene transiently has been developed (Zinyk et al., 1998). A bi-genic system in which Cre recombinase expression is linked to expression from a gene specific to the precursor cell type is present in combination with a reporter that is activated permanently by recombination only when Cre is expressed. Rearrangements leading to reporter expression then occur in the precursor cell and mark its progeny regardless of whether they continue to express the marker gene that caused the initial expression of Cre. This methodology is an effective means of following the fate of a precursor cell in the forward direction. In this method, the stem cell carries both a Cre recombinase vector expressed under the control of the promoter for a known gene and an EGFP (or other) reporter that is activated on Cre expression through a permanent rearrangement within the vector. Hence, transient activation of Cre recombinase within a multipotent precursor induces a rearrangement that results in EGFP expression from the constitutive Pgk promoter in this cell as well as its progeny. Although it is then possible to trace the fate of a precursor cell, there are limitations to the utility of this method for determining the molecular profile of genes expressed within specific cell lineages. Isolation of EGFP expressing cells from a differentiating population will allow recovery of the cell and its progeny but it will not be possible to assign individual genes to specific cell types within the lineage. A second disadvantage is that the precursor specific gene must be known in advance.

Currently there are no methodologies available for rapid characterization of cell lineage genes to obtain a molecular profile of cells of all or most of the changes in gene expression during differentiation. Accordingly, there is an ongoing need to develop new approaches to studying cell lineage specific gene expression.

### SUMMARY OF THE INVENTION

The present invention provides methods and compositions for rapid and comprehensive identification of cell lineage specific genes. The method of the present invention comprises identification of cells destined toward a particular lineage. The steps of the method of the present invention are as follows. The steps required for retrospective gene expression analysis are to: 1) establish an embryonic stem cell line with a recombinase excision-dependent, cell type specific fluorescent protein reporter, 2) use the cell line for recombinase-gene trapping in a library fashion where individual cells will not be derived, but rather ˜10,000-30,000 different insertions will be kept together in a mixed cell culture, 3) isolate cells differentiating towards the marked cell lineage on the basis of fluorescent protein expression using FACS, 4) recover short sequence tags from the trapped genes in a high-throughput fashion and identify the tagged genes.

The steps of the present invention are accomplished by using a vector system comprising the elements for cell lineage targeting, gene trapping and high-throughput analysis of the trapped genes. In one embodiment two vectors are used. One vector is termed herein as the cell lineage targeting vector comprising a cell lineage specific gene, the promoter for that gene, a deletion sequence generally targeted by a recombinase, a selectable marker and a reporter gene. The selectable marker enables the isolation of cells destined toward the selected lineage. A second vector is then introduced into the cells. This vector is termed herein as the gene-trap vector. This vector comprises the elements for the modified serial analysis of gene expression (MAGE), a recombinase and a selectable marker.

In another embodiment, this invention provides a vector system comprising the cell lineage targeting vector and the gene-trap vector for identification and temporal characterization of cell lineage specific genes.

### BRIEF DESCRIPTION OF THE DRAWINGS

FIG. 1 is a schematic diagram of retrospective gene expression analysis for identifying genes expressed within the lineage of a differentiated cell.

FIG. 2 is a schematic diagram of the expected gene identifications based on profiling differentiated cells, cells marked by the currently used forward approach and the reverse gene expression analysis of the present invention. A is a Constitutive gene, B is specific to I, C is specific to II, D is specific to Ia, E is specific to Ib, F is specific to IIa, G is specific to IIb, H is specific to Ib1 and I is specific to Ib2.

FIG. 3 is a schematic representation of the cell lineage targeting vector.

FIG. 4 is a schematic representation of the gene-trap vector.

FIG. 5 is a representation of the modified SAGE approach for primary reporter identification from gene trap cell lines.

FIG. 6 is a representation of the gene-trap vector structure and integration of this vector into an endogenous gene.

FIG. 7 is a representation of the structure of a Cre gene-trap vecotr following integration into an endogenous gene.

FIG. 8 is a representation of the structure of a targeting vector for integration of a Cre-dependent Emerald reporter construct into the 3′ non-translated region of the Synapsis I gene.

FIG. 9 is a representation of fluorescence showing lineage marking with a Cre-gene trap vector with or without Tomaxifen.

FIG. 10a-h is a representation of the fluorescence of humanized EGFP and Emerald fluorescent proteins when expressed from the CMV promoter in 293 cells. FIGS. 10a and 10b are maps of plasmids encoding the humanized GFP (PTRGS-green plasmid) and Emerald (pcDNA3-Emerald plasmid) fluorescent proteins. A histogram the fluorescence intensity is shown in FIGS. 10c and 10d; scatter plots are shown in FIGS. 10e and 10f and the fluorescence and phase contrast images are shown in FIGS. 10g and 10h.

FIG. 11 is a representation of illustrative results from the application of MAGE to a pool of gene trap marked cell lines. Electrophoresed PCR products are shown in Panel A. Electrophoresis of all PCR products in Panel A except the 232 bp is shown in Panel B.

FIG. 12 is a representation of an illustrative plasmid useful for inverse PCR.

### DETAILED DESCRIPTION OF THE INVENTION

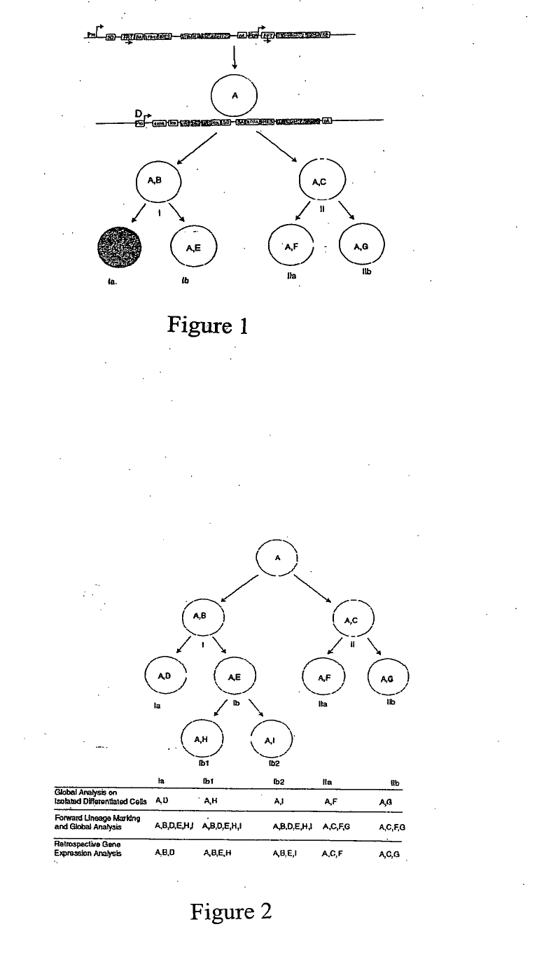

The method of the present invention is based on the following concept. It is desirable to associate a specific differentiated cell type with a molecular profile of all of the changes in gene expression that occurred during its derivation. A conceptual scheme allowing such a profile to be obtained is shown in FIG. 1. In this figure, a simple lineage relationship is shown in which there is a stem cell that can give rise to two multipotent precursor cells I and II. These in turn can lead to the differentiated cell types Ia and Ib or Ia and IIb. Genes are represented by the letters A-G, where all cells express A, B is specific to multipotent precursor I, C is specific to multipotent precursor II and D-G are lineage specific markers for the differentiated cells Ia-IIb as shown in the figure. a molecular profile of the differentiated cell Ia would be constituted by the expression of genes A, B, and D; reflecting the derivation of Ia from I.

In the method of the present invention, a reporter gene expression is driven under control of the differentiated cell specific gene D promoter. Hence, the only cells that will express the reporter gene are the differentiated cells Ia and if the reporter gene is a fluorescent protein, these can be isolated by FACS. However, Ia cells will only express EGFP if a rearrangement in the reporter construct has occurred due to the expression of a recombinase. This occurs only if the gene promoter driving the recombinase was active at some point in the derivation of cell Ia. Further, if recombinase is randomly integrated into different genes in a pool of the starting stem cell population (i.e. in a library fashion), different genes can be sampled for their expression during the derivation of cell type Ia.

A comparison of the information generated by expression profiling methods currently available and the method of the present invention is shown in FIG. 2. The advantage of the retrospective gene expression approach is that it makes it possible to start with a well defined differentiated cell type in which genes have been trapped randomly with a gene-trap vector and in which a cell lineage gene is marked, and look backwards into the gene expression history of just the lineage giving rise to that specific cell type. By this method a through analysis of genes expressed during differentiation all the way back to the point where the genes were trapped, can be done Further, it will capture genes expressed within the lineage even if their expression is only transient and they are no longer expressed in the differentiated cell. If retrospective gene expression data are generated for two or more lineages derived from a common stem cell, it will also be possible to reconstruct the molecular relationship between the lineages from gene expression data obtained by the method of the present invention.

Accordingly, the present invention provides a method for rapid identification of cell lineage specific genes in embryonic stem cells. The method of the present invention comprises introducing into a stem cell line a cell lineage specific gene whose expression, detected by a reporter protein, is dependent upon a recombinase excision event. The recombinase is randomly integrated into different genes. Also present in the vector carrying the recombinase gene are elements of a high throughput analysis for identification of trapped genes. When cells destined toward the selected cell lineage are identified (based on the expression of the reporter protein), the trapped genes (indicating genes expressed at some point during differentiation) can be identified in a high-throughput fashion.

No previous methodology can comprehensively define the history of gene expression, exclusive of those expressed constitutively, within a specific cell lineage. Applied on a broader scale, knowing the history of gene expression for cell types that are derived from a common progenitor will allow establishment of the point at which they diverged and the molecular phenotype of the precursor cell since, at some point in their history, a common set of genes will be identified for the different cell types. This makes it possible to know the molecular details of the relationships between all of the cell types formed following differentiation of ES cells in vitro.

The ability to define genes expressed throughout the history of a given cell lineage would have enormous potential utility. There are numerous applications for this information that would facilitate the objective of defining cell lineages for transplantation. For example, from this set of genes, it is possible to cull those that would provide useful markers for various stages of differentiation towards a desired lineage. The ability to mark precursors at various stages in their differentiation towards different cell lineages for recovery, e.g. with EGFP, makes it possible to test their ability to contribute to a desired tissue. Some of the genes are likely to be potential regulatory molecules. It is possible to use these to direct differentiation to a desired lineage. Cell surface molecules that are specific to early stages of the desired lineage may also be identified. This would offer the possibility of raising antibodies to be used in recovering such cells in the absence of genetic marking and may be particularly important for application to transplantation of such cells in humans. Identification of receptors or components of signal transduction pathways could offer new insights into biologically active agents that may facilitate differentiation of cells towards specific cell lineages. The same set of molecules may be pharmacological targets for modulating the function of stem or progenitor cells.

The present method can also be used in the context of a whole animal. Animals carrying the necessary recombinase dependent reporter protein marked cell type specific gene can be generated (e.g. from the aMHC knock in) as follows. Bone marrow recovered from these cells can be treated essentially as described herein for the stem cells and the cells can then be returned to the animal. Alternatively retroviruses are highly effective in transducing cells in vivo. Hence, at least in those cases where sufficient numbers of stem cells are present for targeting and sufficient numbers of the specific cell lineage to be assessed can be recovered, the gene expression history of the cell type from its stem cell progenitor can be traced in an adult animal. For example, for use in vivo, the Cre-HSVtk recombinase may be advantageous since it would allow elimination of constitutively expressed trapped genes in situ, prior to the start of the gene to lineage linking protocol, through the administration of gancyclovir.

Further, this method can be used for investigating the divergence of genes by identification of temporally expressed genes in two or more lineages. In summary, this technology can be used for fully defining the relationships between cell lineage and gene expression in vitro and, potentially, in vivo.

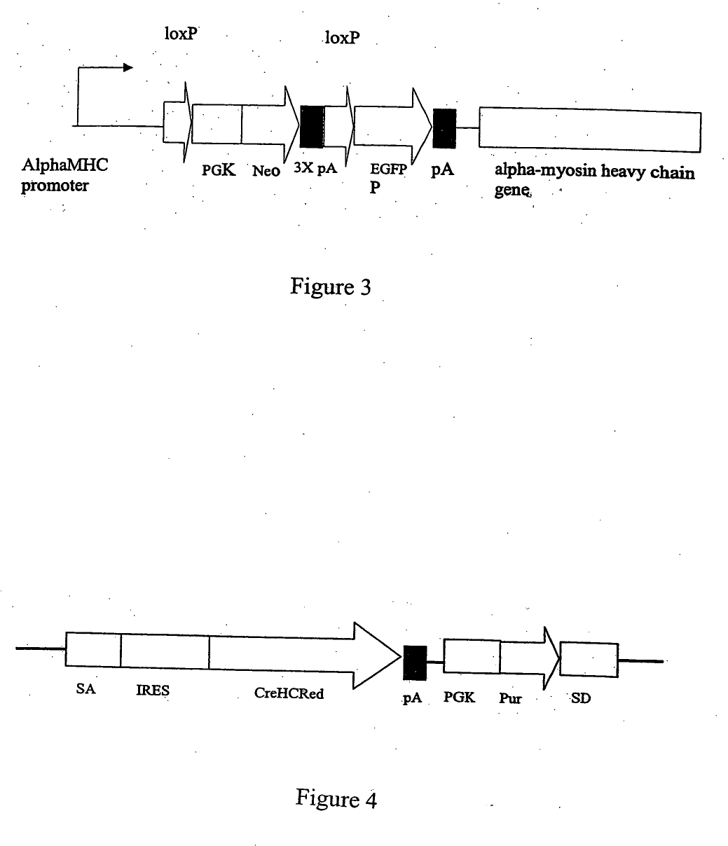

The elements required for the present invention i.e., cell lineage targeting, gene-trapping and high throughput analysis can be introduced in one or more vectors into cells. In one embodiment, the elements are introduced as two separate vectors. The vectors can be introduced in the cell together or in any sequential order. One vector is termed as the cell lineage targeting vector. This vector comprises a cell lineage gene promoter (non-targeted vector for non-targeted recombination), a selectable marker and a reporter gene. Further, a pair of sequences targeted by a recombinase are present flanking a polyadenylation site. In one embodiment, the cell lineage targeting vector comprises a cell lineage specific gene, a selectable marker, a reporter gene and the recombinase target sites flanking the polyadenylation site (targeted vector for targeted recombination). In one embodiment, the selectable marker may be present in the sequence flanked by the recombinase target sites. An example of a targeted vector is shown in FIG. 3. A second vector is termed as the gene-trap vector. It comprises the gene for a recombinase (such as Cre or Flp), a selectable marker and elements for carrying out a rapid identification of trapped genes by the modified serial analysis of gene expression (MAGE). An example of such a vector is shown in FIG. 4.

Any cell lineage specific gene may be used to construct the cell specific lineage targeting vector. Such cell specific genes are well known in the art. For example, to identify the lineage of cardiomyocytes, alpha MHC gene and its promoter can be used. For identifying the lineage of neurons, neuron specific genes such as synapsis or neuron specific enolase can be used. For identifying glial cells, glial fibrillary acidic protein (GFAP) gene can be used. Other examples of cell specific lineage genes are available on the NCBI-GEO web cite which is easily accessible and well known to those skilled in the art.

The selectable marker in the cell specific targeting vector can be based on any positive or negative selection approach. The selectable marker facilitates the selection of host cells transformed or transfected with the vector. Positive selection marker genes are those that encode a protein such as G418, hygromycin, puromycin and blastocidin, which confer resistance to certain drugs and proteins allowing for positive selection. The negative selection includes conditional negative selection, such as HSV-tk in the presence of gancyclovir or acyclovir, and nitroreductase in the presence of metronidezole.

The reporter gene in the cell specific targeting vector is any sequence of DNA that encodes for a protein which is detectable by an assay. In a preferred embodiment, the protein is a fluorescent protein. For example, the green fluorescence protein gene (GFP) isolated from the jellyfish Aequorea Victoria, has become available as a reporter in prokaryotes and eukaryotes. The gfp gene encodes a protein which fluoresces when excited by violet or blue-green light. Variants of GFP are also available. One such variant is the enhanced GFP or EGFP (which is shown in FIG. 1 as being regulated by the promoter of the cell specific lineage gene, αMHC. Another example of a reporter protein is red fluorescent protein (RFP) and yellow fluorescent protein (YFP). Further, proteins which are detectable via histochemical stains (e.g., b-galactosidase, alkaline phosphatase) can be used. Expression of the reporter gene is dependent upon the promoter of the cell lineage specific gene and on the deletion of a recombinase target sequence. In the absence of a recombinase, the recombinase target sequence is not deleted and the transcription of the reporter gene is prevented by the polyA site. The recombinase target sequence depends upon the recombinase in the gene-trap vector. Thus, if the recombinase is Cre, the recombinase target sequence comprises the loxP sites and if the recombinase is FLP, the recombinase target sequence comprises the fit sequences. Further, fusions between two protein that confer the functions of each may also be used (such as b-GEO). It will be recognized by those skilled in the art that reporter proteins other than fluorescent proteins can be alternatively used. Thus, any reporter protein which allows the isolation of cells in which rearrangement by gene-trapping has taken place, can be used. Such reporter proteins include magnetic tags, positive selection such as puromycin, lacZ protein and the like.

In the gene trap vector is present the DNA sequence encoding a recombinase. Examples of recombinase known in the art include Cre and FLP. In one embodiment, the recombinase may be fused to fluorescent protein such as HCRed, or Thymidine Kinase or nitroreductase. In the gene trap vector are also present elements for MAGE (described in U.S. patent application Ser. No. 10/227,719, filed on Aug. 26, 2002, incorporated herein by reference) and as described herein.

The vectors of the present invention can be introduced into suitable host cells by standard methods of transfection including lipofection, precipitation, infection, electroporation, microinjection and the like. Such methods are well known in the art (see for example, see Sambrook et al., Molecular Cloning A Laboratory Manual, 2nd edition, Cold Spring Harbor Laboratory Press, 2001.

In one illustration, the steps of the present method are as follows. A cell lineage specific gene (such as αMHC as an indicator for cardiomyocytes or synapsin I as an indicator of neuronal cells) that can be used for marking lineage is selected. A recombinase (such as Cre or flp) dependent reporter gene is then knocked in to mark the expression of the selected cell lineage gene. The sequence deleted by the recombinase also comprises a selectable marker such as a PGK driven neomycin resistance gene. An example of such a vector is shown in FIG. 3. By the use of this vector, cells destined toward the selected lineage are identified by the selectable marker and these cells can be isolated.

In the next step, cells are tranfected with a gene-trap vector comprising a recombinase. The gene-trap vector also comprises elements for a high throughput detection assay. Preferably cells without base level recombinase are selected. In one embodiment, an inducible recombinase (such as tamoxifen dependent Cre recombinase) can be used. An example of a gene-trap vector is shown in FIG. 4.

The expressed genes at various stages of differentiation can be characterized by using the modified serial analysis of gene expression (MAGE) technique. Key to the ability of the present technology to provide a global profile of lineage specific gene expression is a means of identifying gene trap insertion sites efficiently. Elements allowing high efficiency acquisition of sequence tags that identify such sites are incorporated within the splice junctions of the Cre-Gene Trap Vector to be used. It is this feature of the gene trap vector that permits a modified version of the Serial Amplification of Gene Expression (SAGE, Velculescu et al., 1995) technology to be utilized in identification of trapped genes in a high throughput format. This technology is referred to as MAGE and is described in detail below.

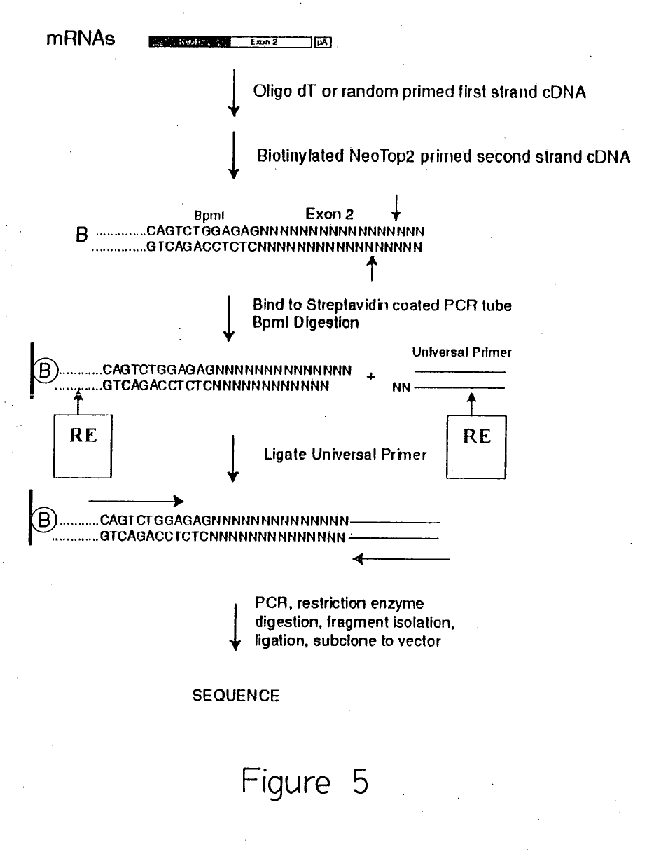

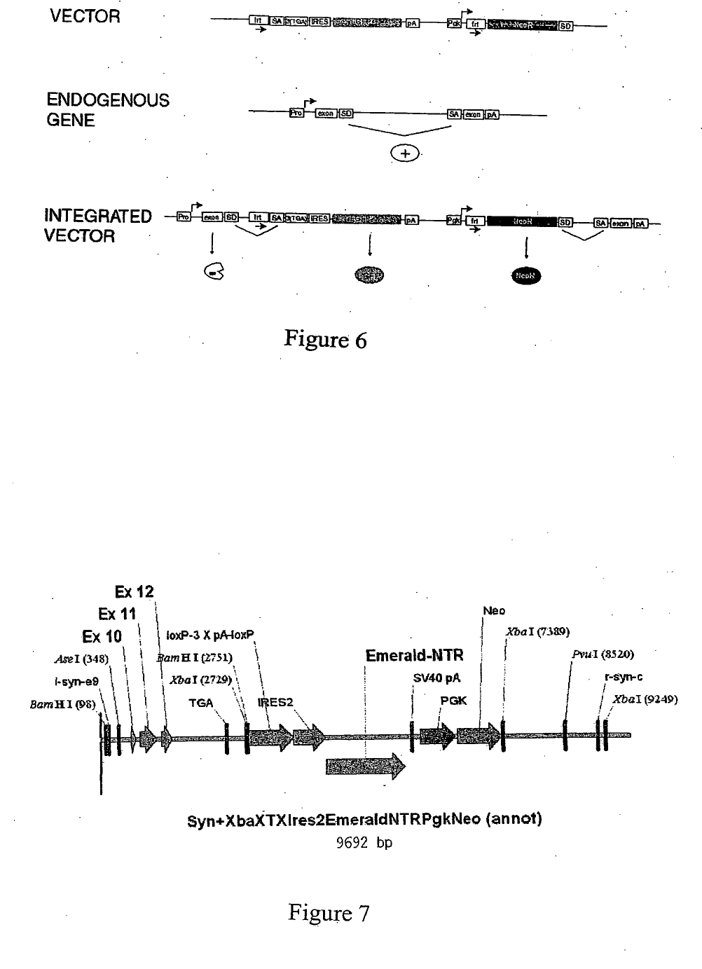

The Modified SAGE technology (MAGE) is a high throughput method for the identification of sequence tags resulting from gene trap vector integration events. The basis of this technology is shown in FIG. 5. The first element on which it depends is the incorporation of type IIS endonuclease restriction (such as BsgI and BpmI) recognition sequences adjacent to the splice acceptor and splice donor elements within the HTP gene trap vector. These type IIS restriction endonucleases have the property that each cleaves the DNA at a position 16 nucleotides adjacent to the recognition sequence where the composition of the 16 nucleotides is irrelevant. Other examples of type IIS sites are BsmFI, MmeI and FokI. Using the example of BsgI and BpmI, as shown in FIG. 6, we have taken advantage of this property to allow the amplification of either 15 or 14 nucleotides of the endogenous gene sequence adjacent to the SA and SD elements of the gene trap vector, respectively. This allows differential amplification of endogenous gene sequence from cDNAs to messages that result from transcripts initiating from the endogenous gene promoter when BsgI is used or the Pgk promoter when BpmI is used. Hence, in the case of BsgI, the resulting products will reflect the relative expression level from the marked gene when assaying mixed pools while BpmI will result in relatively even levels of amplification products. This becomes important below.

Following this, bits of unknown sequence information can be identified because these are separated by repeats of a known sequence. In the present application, this is accomplished by ligating the PCR products with the aid of a restriction endonuclease cleavage site present in both the universal primer and adjacent vector sequence. The ligated strings of sequence tags are then cloned and sequenced. Thus, each sequence tag representing each member present in a pool of marked genes regardless of the absolute expression level, can be sequenced. Since transcripts expressed from the Pgk promoter will be present at relatively equal levels, use of the SD junction fragments is optimal. Data similar to the following sequence string can be obtained: 5′TCTAGACAGTCTGGAGAAGNNNNNNNNNNNNNNTCTAGACAGTCTGGA GAGNNNNNNNNNNNNNNTCTAGACAGTCTGGAGAGNNNNNNNNNNNN NNTCTAGANNNNNNNNNNNNNNCTCTCCAGACTGTCTAGACAGTCTGGA GAGNNNNN3′ (SEQ ID NO:1). Each repeating unit is 32 nucleotides long and contains 16 nucleotides that are derived from a discrete gene trap event (the splice donor AG plus 14 as underlined) and can be used to identify the insertion site. Inversion of the repeats is possible; however, this event is easily recognized.

In one embodiment, to address the issue of different RNAs being expressed at different levels which can skew representation of certain RNAs, instead of RT PCR, inverse PCR can be used. Inverse polymerase chain reaction (IPCR) is an extension of the polymerase chain reaction that permits the amplification of regions that flank any DNA segment of known sequence, either upstream or downstream (see U.S. Pat. No. 4,994,370, specifically incorporated herein by reference in its entirety). The essence of IPCR is that, by circularizing a restriction enzyme fragment containing a region of known sequence plus flanking DNA, PCR can be performed using oligonucleotides whose sequence is taken from the single region of known sequence and oriented with respect to one another such that their 5′ to 3′ extension products proceed toward each other by going “around the circle” through what originally was flanking DNA. This leads to the amplification of DNA strands containing what was originally flanking DNA. The advantage of a technique such as IPCR, with respect to the current invention, is that using a single primer set one may amplify a representative sample of insertion junctions from a particular group of individuals.

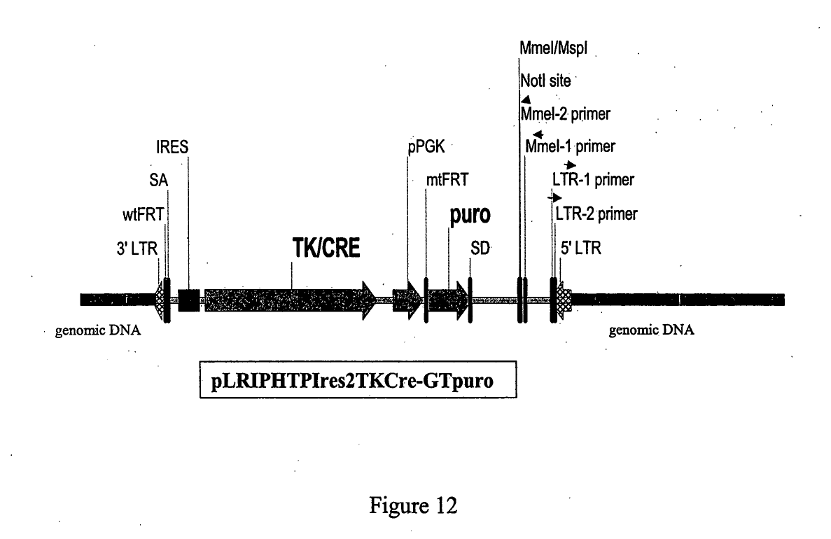

An illustrative example of inverse PCR is provided below. An illustrative plasmid is shown in FIG. 12. For inverse PCR, genomic DNA is pooled from the cells expressing the reporter gene. An aliquot is digested with MspI The digested DNA is ligatged to circularize and amplified using an LTR primer and biotinylated MmeI-1 primer. The amplicon is immobilized on a strepavidin tube and digested with MmeI to expose genomic tags. The tags are ligated to a universal oligo, amplified and purified by HPLC. The tags can then be concatamerized and ligated into plasmid vectors.

In one embodiment, the cloning vector containing two different cloning ends (in one embodiment, SacI and NotI) are used. The tag concatamers are derived by ligation of the tags at a dilute concentration in the presence of 0.5:1 Molar ratio of each of two non-phosphorylated DNA adapter molecules (in this embodiment the adapters would contain both XbaI overhangs for ligation to the tag cloning ends, while on the opposite end one adapter would have a NotI overhang and the other would have a SacI overhang). The use of non-phosrphorylated adapters minimizes the formation of any inhibitory side reaction products. The use of different ends on the cloning vector allows the more efficient directional cloning of the concatamers. The adapters are added to the reaction before addition of the tag monomers in order to maximize monomer tag addition while simultaneously minimizing self ligation of monomers into mini DNA circles (which would result in a loss of critical material). The ligation of an adapter to one end of a concatamer prevents circularization of that molecule while allowing continued addition of monomer tags to the other end until that too becomes ligated to an adapter. Those concatamers which have different overhangs at each end (e.g. SacI at one end and NotI at the other) will clone with high efficiency into the recipient vector.

This invention is further described in the Examples presented below which are intended to be illustrative and not restrictive.

### EXAMPLE 1

In a further illustration of this invention, the method can be carried out in three steps. Initially, an embryonic stem (ES) cell is created in which a specific cell lineage is marked by knocking in a Cre-mediated recombination dependent EGFP vector. Second, a highthroughput (HTP)-gene-trap vector that both marks genes with a tamoxifen dependent Cre recombinase and allows detection of expression of the marked gene by expression of hcRFP (or alternatively allows negative selection of the gene by expression of herpes simplex virus thymidine kinase), is created. Finally, the ES cell line is utilized in conjunction with the gene trap vector as follows to identify genes expressed at different stages in the differentiation towards the marked cell lineage.

Following transfection of the ES cell line with the gene trap vector and selection for its integration, cells can be first sorted for lack of RFP expression, induced to differentiate, and treated with tamoxifen at various times. Due to Cre mediated re-arrangement of the EGFP reporter, cells that carry a gene trap event in a gene that is expressed in the marked cell lineage will, ultimately, express EGFP. These cells can be recovered by FACS, and the genes incorporating the gene trap vector can be determined using a HTP-sequence tag identification technique (MAGE).

1) ES cell line derivation. To establish the initial knock in ES cell line a vector of the structure shown in FIG. 3 and allowing Cre dependent activation of EGFP expression from a cell lineage specific gene is created. The example shown in FIG. 3 uses the AMHC promoter which is useful for marking cardiomyocytes. The construct can be electroporated into W4 ES cells and colonies can be selected in G418 and scored for correct integration at both the 5′ and 3′ ends. Sufficient colonies are scored to establish between 3 and 5 independent lines. One or more lines are selected for further use on the basis of efficient differentiation towards cardiomyocyte lineages.

2) Gene trap vector construction. A gene trap vector incorporating the sequences necessary for both HTP sequence tag acquisition by MAGE and reverse orientation retroviral packaging can be constructed as shown in FIG. 4. This vector carries a tamoxifen dependent Cre recombinase fused to the fluorescent protein HCRedl. Hence, on integration into an endogenous gene, expression from the gene will both lead to Cre expression and be detectable through fluorescence in the Red channel. The vector is packaged and utilized to transduce the AMHC conditional EGFP marked cell line derived as described above. Cells are selected in the presence of puromycin and the resulting colonies are pooled. Approximately 10,000 to 30,000 colonies are desired, which using an appropriately tittered viral stock, are achievable using approximately 10×15 cm culture dishes. Colonies can be pooled and used in the protocol described below.

3) Identification of lineage specific genes. The third and informative component of this technology is to recover lineage specific (in the current example cardiomyocyte) genes from the pool of gene trapped cells. This is accomplished as follows. First, cells are sorted for those that do not express detectable levels of RFP. It is important here that cells that are negative for RFP do not express Cre recombinase at functional levels. Hence, a sample of the non-RFP expressing population is grown in the presence of tamoxifen (for 48 hours) and assessed for recombination at the αMHC conditional EGFP gene by PCR. In the event that significant levels of recombination are occurring, it can be concluded that RFP is not a sufficiently sensitive marker and the gene trap vector to incorporate and Cre-HSVtk fusion can be reconstructed. In this case cells can be selected against HSVtk expression using gancyclovir and the efficacy of this selection can be confirmed by analysis of recombination at the αMHC conditional EGFP gene. If HSVtk is not a sufficiently sensitive negative selection, progressively disabled versions of Cre recombinase through sub-optimal codon usage substitutions can be created as is known to those skilled in the art. The effect of the negative selection at this step is to remove from the gene trap marked cell population those genes that are active in uninduced cells.

A second level of characterization of the non-expressing cells is performed to define the full range of tagged genes. For this analysis MAGE is performed from the splice donor side of the vector on the pool of un-induced non-RFP expressing cells. If we have tagged the desired 30,000 cells, there should be a corresponding 30,000 sequence tags to acquire or 30,000×32 bp of non-redundant sequence. This is 960,000 bp. Each sequence run is expected to yield about 500 bp of information so approximately 1,900 sequence reactions or 20 microtitre plates of sequence will be required. This can be performed on the MegaBase capillary sequencer within 3-4 days. For approximately 3× coverage to detect 95% of the tagged genes, the estimated time to complete the sequencing is about 3 weeks. Establishing the full complement of genes that could be detected provides an important base line against which to measure the subset that is detected in the desired cell lineage as described below.

Following sorting for cells that do not express RFP (or, alternatively, grow in the presence of gancyclovir), these cells will be induced towards cardiomyocyte lineages using an optimized induction regime. Tamoxifen is introduced into the culture media at progressively later stages of induction in different cultures where, initially, selected intervals (such as 1 day intervals) can be used. In each culture, cells are treated with tamoxifen (e.g., for 24 hours). During this treatment, Cre expression from active genes will allow recombination at the cell lineage specific conditional EGFP gene. Recombination will occur in all cells that carry a marked gene that is active during the time tamoxifen is added regardless of whether they are destine to become, in the example here, caridomyocytes or alternative lineages or whether the gene remains active even within the cardiomyocyte lineage. Regardless of the time of tamoxifen addition, cells will be cultured through 8 days at which point activation of the αMHC gene should occur within all cells in the cardiomyocyte lineage. Only those cells that expressed Cre from a gene that was marked by a gene trap event and expressed in the cardiomyocyte lineage during the time tamoxifen was active will activate EGFP expression. Cells are then trypsinized and sorted using FACS to recover these cells. Sequence tags from the marked genes will be identified using MAGE from the splice donor BpmI site. These sequence tags will identify sets of genes expressed specifically in the cardiomyocyte lineage at various points in its derivation from ES cells. If sufficiently large numbers of gene trap events are scored, this method can be used to define and temporally order the expression of the large majority of genes that are specific for the cardiomyocyte lineage.

### EXAMPLE 2

This example describes the construction of another cell lineage specific targeting vector. In this example, a Cre-recombinase excision dependent Synapsin I-specific Emerald reporter construct is described. The neural specific gene, Synapsin L is known to be expressed in neurons derived from retinoic acid (RA) treated ES cell cultures (Finley et al; 1996). The targeting construct shown in FIG. 7 was prepared and an AseI to PvuI fragment was isolated and electroporated into the W4 ES cell line (Taconics).

G418 resistant colonies can be amplified and assessed for correct targeting into the Synapsin I gene by BamHI and XbaI digestion and Southern blotting to assess correct targeting at the 5′ and 3′ ends respectively. In this illustration, the targeting construct comprised the fluorescent protein Emerald. The expression of this Emerald from the Synapsin I promoter following Cre recombinase mediated excision of the triple polyA stop signal (or lox-STOP-lox cassette, abbreviated XTX) allows the identification and isolation of these cells.

### EXAMPLE 3

This example describes the Construction and transduction efficiency of a lenti-viral based gene trap vector. An example is shown in FIG. 6. Elements required for gene-trapping functions include a reporter gene (here EGFP) downstream of a splice acceptor such that, on integration into an intron of an endogenous gene, the reporter will become spliced into the endogenous message allowing its expression. In most cases this also disrupts function of the endogenous gene. An internal ribosome entry site (IRES) is placed 5′ to the EGFP sequence to allow its expression regardless of the reading frame of the endogenous transcript. To insure that ribosomes initiating from the endogenous transcript start codon will not occlude the IRES or result in a fusion protein, a series of translation termination codons are placed 5′ to the IRES. The vector also carries a selectable marker (here neo) driven from a constitutive promoter (Pgk) and followed by a splice donor to allow selection of stably transfected cell lines on integration into an endogenous gene. Not shown are viral packaging sites that allow reverse orientation packaging into a self-inactivating (SIN) lentivirus.

Elements allowing high efficiency acquisition of sequence tags are incorporated within the splice junctions. This is a key feature of the vector that permits this technology to be utilized in identification of trapped genes in a high throughput format.

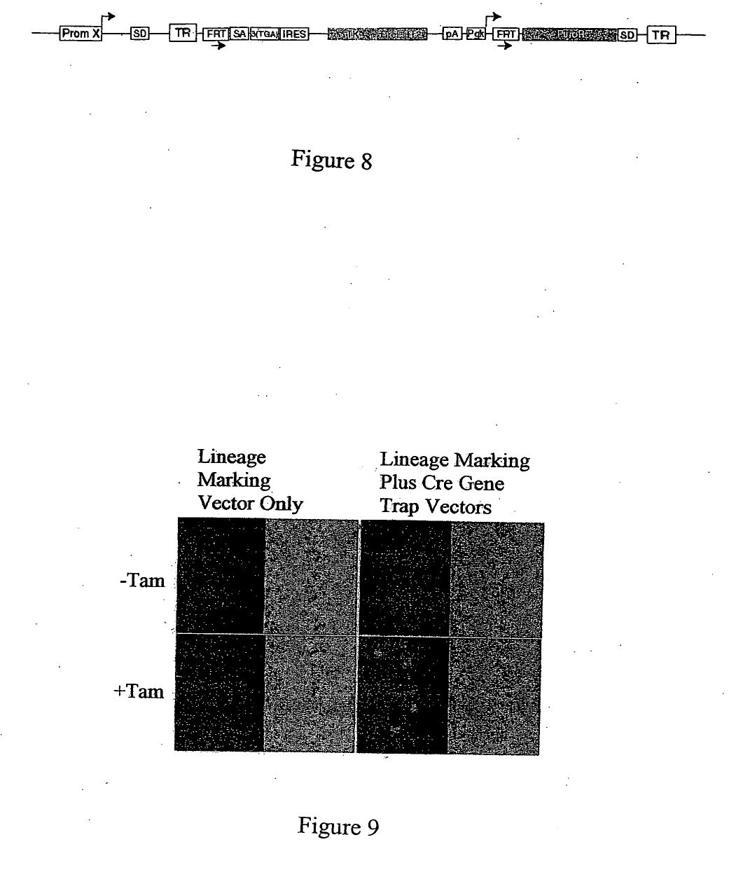

In this embodiment, the following modifications were made (shown in FIG. 8). First, the fluorescent protein reporter has been substituted for an HSVtk/CreERT2 cassette, which encodes a fusion protein between the herpes simplex virus thymidine kinase gene and a tamoxifen dependent Cre recombinase (Feil et al., 1997). Although only the Cre recombinase component is required for the core gene-trap methodology, variations of the method may take advantage of either the HSVtk or tamoxifen dependence of Cre in this fusion protein and these variations are discussed in the experimental methods section. Second, to allow use in cells that already carry a neo resistance gene, the neo gene has been replaced with a puromycin resistance gene (PURO) in the present construct. Other elements, including the SA and SD sequences modified for high-throughput sequence tag analysis are maintained in the Cre gene trap vector.

The function of both the Cre-recombinase and the loxStoplox (XTX) elements used in the Cre-gene-trap-vector and the Cre-dependent-Synapsin-I-Emerald-reporter constructs, respectively, were tested as shown in FIG. 9. Here, the Pgk promoter was used to drive expression from either the gene trap vector as shown in FIG. 8 or an EGFP reporter containing the XTX element used in the Synapsin construct as shown in FIG. 7. NIH 293 cells were tranfected with either the lineage marking vector alone or this vector plus the Cre gene trap vector and grown in either the presence or absence of tamoxifen (FIG. 8). No expression of EGFP from the lineage-marking vector was found in the absence of the Cre gene-trap vector. In the presence of the Cre gene trap vector and tamoxifen, but not in the absence of tamoxifen, robust EGFP expression was observed in co-transfected cells.

### EXAMPLE 4

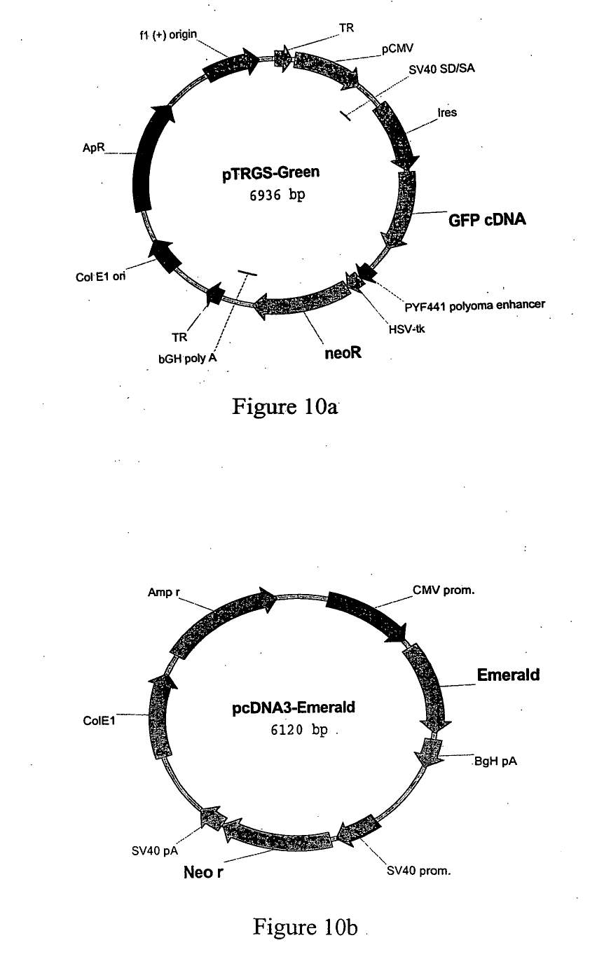

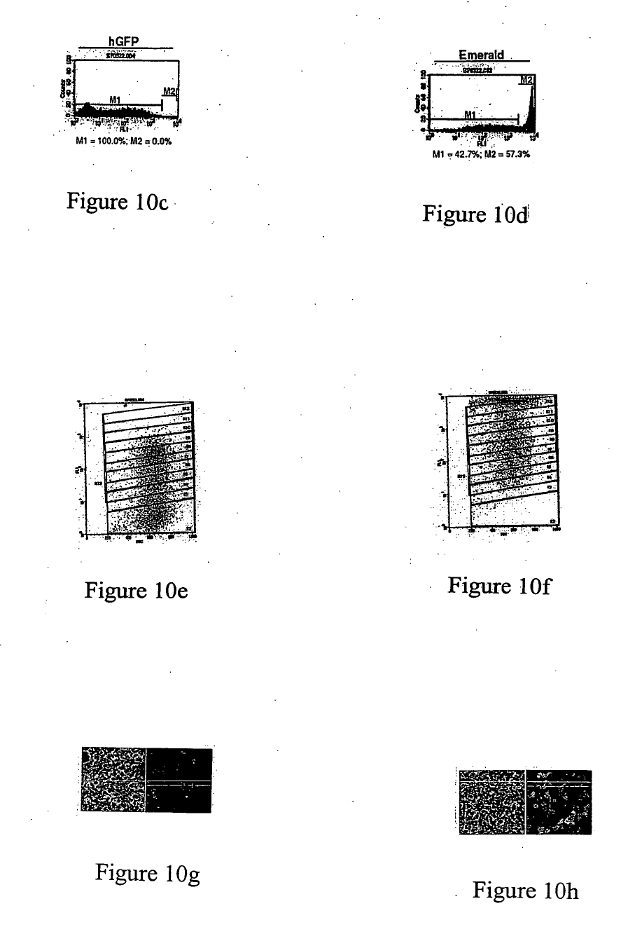

This example describes the isolation of fluorescent protein expressing cells by FACS. An important component of this invention is the ability to recover reporter gene expressing cells in the absence of significant levels of non-expressing cells. As an illustration of this embodiment, a comparison of the fluorescence of humanized GFP and Emerald fluorescent proteins when expressed from the CMV promoter in HEK293 cells was carried out. The plasmids used are shown in FIG. 10a and 10b. The results are shown in FIG. 10c-h. FIGS. 10c,e and g are hGFP while FIGS. 10d, f and h are Emerald. HEK293 cells were transiently transfected with each plasmid using lipofectamine 2000. 36 hours later cells were trypinized and sorted on a BD FACSVantage instrument. FIGS. 10c and d are histograms of fluorescence intensity (FLI) on a log scale; FIGS. 10e and f are Scatter plot log fluorescence against forward scatter (FSC); FIGS. 10g and h are fluorescence images of transfectants obtained just prior to sorting using a Nikon Eclipse TE300 fluorescent microscope and a SPOT diagnostics camera. Images were processed using SPOT diagnostics software version 3.0.4.

### EXAMPLE 5

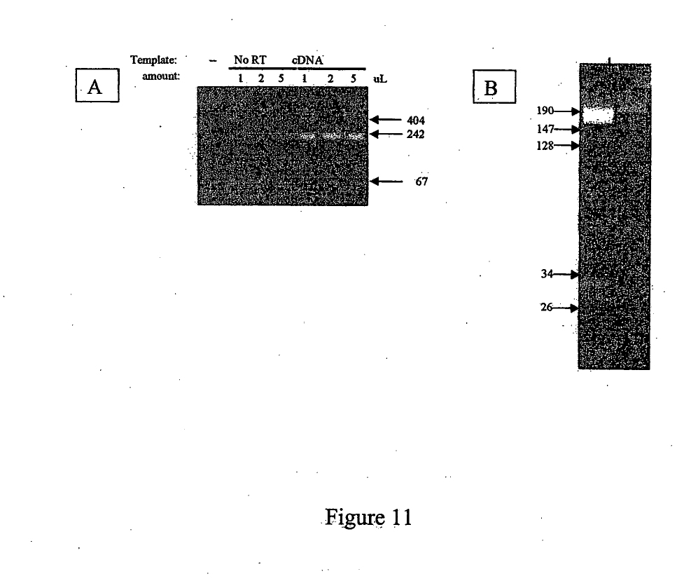

This example describes a high-throughput vector dependent sequence tag recovery. As proof of principle, the generation and identification of Sequence Tags from trapped genes by MAGE from the splice donor junctions present in a small pool of P19 EC cell gene trap lines was performed. RNAs were isolated using GIT/phenol extraction and polyadenylated messages were selected on oligo dT cellulose by standard methods. First strand cDNA synthesis primed with oligo dT was performed using superscript II (Invitrogen) using standard conditions. A control sample in which reverse transcriptase was omitted was also prepared. RNA was hydrolyzed using NaOH. NaOH was neutralized and cDNAs were recovered by ethanol precipitation. Second strand synthesis was rimed using Biotinylated neotop2 primer (5′-B-CCGCTTTTCTGGATTCAT-3′-SEQ ID NO:2) and extended using the large fragment of E. coli DNA polymerase. Double stranded cDNA was degiested with BpmI and incubated with streptavidin coated PCR tubes for 3 minutes at 37C. Following binding tubes were washed times with 150 ul of 150 mM NaCl in TE (10 mM Tris-HCl, pH 7.5, 1 mM EDTA). The MAGE universal adapter (5′-TCTAGAGGACTGCGTGGGCGA-3′-(SEQ ID NO:3); 5′-CCTCGCCCACGCAGTCCTCTAGANN-3′-(SEQ ID NO:4) (16.6 nmoles) was added in 50 ul of ligation buffer plus 2 ul of T4 ligase and tubes and tubes were incubated for 2 hours at 15C. Following 2 washes as previously, 1 ul of 50 uM of each MAGE PCR primer (5′-CCTCGCCCACGCAGTCCTC-3′ (SEQ ID NO:5), 5′CGGCTGGGTGTGGCGGAC-3′-(SEQ ID NO:6)) was added in 100 ul of Platinum Taq (Invirogen) PCR reaction buffer containing 0.2 mM of each of dATP, dGTP, dCTP and dTTP, 2 mMMgCl₂ and 0.5 units of Platinum Taq polymerase. Thermal cycling was performed where 35 cycles of 94C for 0.75 minutes, 60C for 0.75 minutes and 72C for 0.75 minutes. Samples of the resulting PCR products were electrophoresed on an agarose gel as shown in Panel A of FIG. 11. The predicted band of 232 bp is present in the lanes in which reverse transcriptase was present in the initial cDNA synthesis reaction but is absent from lanes whre reverse transcriptase was initted. The remaining PCR products were pooled, digested with XbaI and electrophoresed on an 8% polyacrylamide as shown in Panel B. The predicted 32 nt fragment containing the sequence tags was isolated and incubated in 20 ul of T4 DNA ligation buffer for between 20 and 60 minutes and used for transformation of competent HB101. Transformed colonies of HB101 were selected and used in preparation of DNA for sequencing by standard techniques. The data from the sequencing is shown in Table 1.

TABLE 1

| | |
|---|---|
| Sequence Tag | Gene |

| | |
|---|---|
| GGCGACACGCGCACCT | erythroid differentiation |
| (SEQ ID NO: 7) | regulator |
| AGGGAGGAGATGTAGT | IMAGE: 2631676 mRNA |
| (SEQ ID NO: 8) | |
| ACTACATCTCCTCCCT | unigene cluster ENC1 |
| (SEQ ID NO: 9) | |
| GGGTTGTCTTCACTCT | unigene cluster, IAP-related |
| (SEQ ID NO: 10) | retroviral elements |
| CCCAGGATGTATAGCT | IMAGE: 3416396 |
| (SEQ ID NO: 11) | |
| AGTCTGGAAACCTGTC | cDNA clone H3123F115 |
| (SEQ ID NO: 12) | |
| ACTACATCTCCTACCT | cDNA clone 9330145B06 3′ |
| (SEQ ID NO: 13) | |
| AGGTAAGTGCTGCTGC | mouse EST, UI-M-BH2.3 aog-c-05-0- |
| (SEQ ID NO: 14) | UI.sI |
| AGGTAAGTACAAGCTG | mouse EST uj60b10.x1 |
| (SEQ ID NO: 15) | |
| ACCTCAGTTGATTCCT | cDNA clone A430056F03 3′ |
| (SEQ ID NO: 16) | |
| AGCTTTGAATTCATGA | hsp84-1 |
| (SEQ ID NO: 17) | |
| TTTCTCAGGGTAGCCT | albumin |
| (SEQ ID NO: 18) | |
| TCCGCTCAATGTACCT | unknown |
| (SEQ ID NO: 19) | |
| AGGGTTGGACTCAAGG | unknown |
| (SEQ ID NO: 20) | |
| ACTACATCTCCTCCGT | unknown |
| (SEQ ID NO: 21) | |
| CACGGGGGCGGAGCCT | unknown |
| (SEQ ID NO: 22) | |
| AGGTAACCCTGTGCTG | unknown |
| (SEQ ID NO: 23) | |
| AGGTAGACTACTTGTG | unknown |
| (SEQ ID NO: 24) | |
| CGCCATCGCTCTAGCT | unknown |
| (SEQ ID NO: 25) | |

Sequencing revealed that the concatamers consisted of the predicted vector/universal primer sequences separated by 16 nucleotide long tags. Blast searches of the tags revealed seven unknown sequences (i.e. not present in the NCBI mouse EST or non-redundant sequence databases) and twelve known sequences comprising predicted exons from albumin, HSP84, actin binding protein, erythroid differentiation regulatory protein and others.

### EXAMPLE 6

In another illustration of the embodiment, further sequence tag sequences were generated as described in Example 5. The sequences are provided below in Table 2.

TABLE 2

| well | | |
|---|---|---|
| no. | Sequence Tag | Gene |
| A2 | AGGCTCATCAGCTGAC | RIKEN cDNA 1600014E20 (chr. |
| | (SEQ ID NO: 26) | 2, 2F2) embryo E6.5-E8.5, |
| | | placenta |
| A4 | AGGTGTGGATCCAGAG | RIKEN cDNA 9130023F12 gene |
| | (SEQ ID NO: 27) | (chr. 11) mammary gland; |
| | | Tcell; head; urinary bladder; |
| | | thymus; lung; brain; spon- |
| | | taneous tumor, metastatic to |
| | | mammary. Stem cell origin; |
| | | neural retina |
| A7 | AGGTAGGTCTCGATCT | no identification |
| | (SEQ ID NO: 28) | |
| A8 | AGAATACGATACCCAG | no identification |
| | (SEQ ID NO: 29) | |
| B7 | AGGGCATTTCTATTAC | no identification |
| | (SEQ ID NO: 30) | |
| C4 | AGCTAATCACCAAAGC | RIKEN cDNA C330027G06 gene |
| | (SEQ ID NO: 31) | (chr. 3, 3G2) numerous |
| | | tissues |
| C6 | AGGATCCACTGTTTAC | no identification |
| | (SEQ ID NO: 32) | |
| D12 | AGGTCTGGAGTAACCA | partial BpmI digest product, |
| | CACATAGATGTTAGTT | phospholipase c-like 2 gene, |
| | AAGAGAGAAAAGTAA | opp ori relative to gene, prob |
| | CTGGAGACTTCCTCAC | cryptic splice (chr. 9) |
| | AATGAG | |
| | (SEQ ID NO: 33) | |
| E10 | AGGTCCTGCCTCAGCA | repetitive |
| | (SEQ ID NO: 34) | |
| F2 | AGGACTGATTGTGGTG | G protein-coupled receptor 39 |
| | (SEQ ID NO: 35) | (Gpr39, chr. 1, 1E3) heart; |
| | | testis; embryo; fetus; whole |
| | | brain; visual cortex* |
| F3 | AGATAAGTTTGTTCTG | no identification |
| | (SEQ ID NO: 36) | |
| F9 | AGGTCCAGTAGGGACC | no identification |
| | (SEQ ID NO: 37) | |
| G6 | AGGTATGATGACAGGT | no identification |
| | (SEQ ID NO: 38) | |
| H3 | AGGTGTACGAATGCGA | no identification |
| | (SEQ ID NO: 39) | |

*Lineage restricted gene trapped by XTX - Gpr39

From the foregoing, it will be obvious to those skilled in the art that various modifications in the methods described herein can be made without departing from the spirit and scope of the invention. Accordingly, the invention may be embodied in other specific forms without departing from the essential characteristics thereof. The embodiments and examples presented herein are therefore to be considered as illustrative and not restrictive.

### REFERENCES

1. Ramalho-Santos M, Yoon S, Matsuzaki Y, Mulligan R C, Melton D A. “Stemness”: transcriptional profiling of embryonic and adult stem cells. Science. 2002 Oct. 18; 298(5593): 597-600
2. Ivanova N B, Dimos J T, Schaniel C, Hackney J A, Moore K A, Lemischka I R. A stem cell molecular signature. Science. 2002 Oct. 18; 298(5593): 601-4.
3. Sano H, Nakamura A, Kobayashi S. Identification of a transcriptional regulatory region for germline-specific expression of vasa gene in Drosophila melanogaster. Mech Dev. 2002 March; 112(1-2): 129-39
4. Xian H Q, McNichols E, St Clair A, Gottlieb D I. A subset of ES-cell-derived neural cells marked by gene targeting. Stem Cells. 2003; 21(1): 41-9.
5. Zinyk D L, Mercer E H, Harris E, Anderson D J, Joyner A L. Fate mapping of the mouse midbrain-hindbrain constriction using a site-specific recombination system. Curr Biol. 1998 May 21; 8(11): 665-8.
6. Finley M F, Kulkarni N, Huettner J E. Synapse formation and establishment of neuronal polarity by P19 embryonic carcinoma cells and embryonic stem cells. J. Neurosci. 1996 Feb. 1; 16(3): 1056-65.
7. Velculescu V E, Zhang L, Vogelstein B, Kinzler K W. Serial analysis of gene expression. Science. 1995 Oct. 20; 270(5235): 484-7.
8. Feil R, Wagner J, Metzger D, Chambon P. Regulation of Cre recombinase activity by mutated estrogen receptor ligand-binding domains. Biochem Biophys Res Commun. 1997 Aug. 28; 237(3): 752-7.

1. A method for identifying genes expressed during differentiation of a cell comprising the steps of:
a) integrating into a site in the genome of a host cell, a cell lineage targeting vector comprising, a pair of recombinase recognition sites flanking one or more polyadenylation sites, a first selectable marker placed downstream of or between the two recombinase recognition sites, a reporter gene placed downstream of the recombinase recognition sites, and a cell lineage specific gene promoter placed upstream of the recombinase recognition sites or a cell specific lineage gene placed downstream of the recombinase recognition sites,
b) amplifying cells generated from the host cell;
c) integrating into the genome of a plurality of the amplified cells, a gene-trap vector comprising a splice acceptor, a type IIS restriction endonuclease cleavage site, a recombinase, one or more polyadenylation sites, a second selectable marker and a splice donor;
d) allowing the cells to differentiate;
e) isolating cells in which the reporter gene is expressed indicating expression of the cell lineage specific gene;
f) identifying trapped genes in the isolated cells.

2. The method of claim 1, wherein the identification of trapped genes in the isolated cells comprises the steps of a) preparing from isolated cells in 1e), concatamers comprising portions corresponding to trapped genes in the isolated cells;
b) sequencing the concatamers to identify trapped genes;
wherein the each trapped gene is indicative of a gene expressed during differentiation.

3. The method of claim 2, wherein the portions of trapped genes are amplified by inverse PCR.

4. The method of claim 2, wherein the portions of trapped genes are amplified by RT PCR.

5. The method of claim 1, wherein the step of identifying the trapped genes in step f) comprises the steps of:
a) preparing mRNA from cells in which the fluorescent reporter is expressed in d);
b) synthesizing a first and second cDNA strands from the mRNA;
c) digesting with type IIS restriction endonucleases to produce Assay Tags wherein each Assay Tag comprises a portion of a trapped gene and a portion of the gene-trap vector;
d) concatenating the Assay Tags;
e) amplifying and sequencing the concatamers to identify the sequence of the portion of the trapped gene.

6. The method of claim 5, wherein the second DNA strand is biotinylated.

7. The method of claim 1, wherein the Type IIS restriction endonuclease is selected from the group consisting of BsgI, BpmI, BsmFl, MmeI and FokI.

8. The method of claim 1, wherein the reporter protein is a fluorescent protein.

9. The method of claim 8, wherein the fluorescent reporter protein is EGFP.

10. The method of claim 1, wherein the recombinase is Cre or FLP.

11. The method of claim 10, wherein the recombinase is fused to thymidine kinase or nitroreductase.

12. A method for identifying genes expressed during differentiation of a cell comprising the steps of:
a) integrating into a site in the genome of a host cell, a cell lineage targeting vector comprising a pair of recombinase recognition sites flanking one or more polyadenylation sites, a first selectable marker, a reporter gene, and a cell lineage specific gene promoter or a cell lineage specific gene, wherein recombinase based excision allows the expression of the reporter gene;
b) amplifying cells generated from the host cell;
c) integrating into a plurality of the amplified cells, a gene-trap vector comprising a splice acceptor, a type IIS restriction endonuclease cleavage site, a recombinase, a second selectable marker, and either a splice donor or a polyadenylation site, wherein integration of the gene-trap vector into an endogenous gene allows the recombinase to be produced and also incorporates a type IIS endonuclease site into the endogenous gene.
c) allowing the host cells to differentiate;
d) isolating cells in which the reporter gene is expressed indicating expression of the cell lineage specific gene;
e) digesting DNA from the isolated cells to form fragments comprising portions of trapped genes;
f) concatenating and sequencing the fragments comprising portions of trapped genes.

13. The method of claim 12, wherein the fragments of DNA comprising portions of trapped genes are amplified by inverse PCR.

14. The method of claim 12, wherein the fragments of DNA comprising portions of trapped genes are amplified by RT PCR.

15. A method for identifying genes expressed during differentiation of a cell comprising the steps of:
a) integrating into a site in the host cell of a genome, a cell lineage targeting vector comprising a pair of recombinase recognition sites flanking one or more polyadenylation sites, a first selectable marker, a reporter gene, and a cell lineage specific gene promoter or a cell lineage specific gene, wherein recombinase based excision allows the expression of the reporter gene;
b) amplifying cells generated from the host cell in a)
c) integrating into a plurality of the amplified cells, a gene-trap vector comprising a splice acceptor, a type IIS restriction endonuclease cleavage site, a recombinase, a second selectable marker, and either a splice donor or a polyadenylation site, wherein integration of the gene-trap vector into an endogenous gene allows the recombinase to be produced and also incorporates a type IIS endonuclease site into the endogenous gene.
c) allowing the host cells to differentiate;
d) isolating cells in which the reporter gene is expressed indicating expression of the cell lineage specific gene;
e) preparing mRNA from cells in which the reporter gene is expressed in d);
f) synthesizing a first and second cDNA strands from the mRNA;
g) digesting with type IIS restriction endonucleases to produce Assay Tags wherein each Assay Tag comprises a portion of a trapped gene and a portion of the gene-trap vector;
h) concatenating the Assay Tags;
i) amplifying and sequencing the concatamers to identify the sequence of the portion of the trapped gene.

16. The method of claim 15, wherein the second DNA strand is biotinylated.

17. The method of claim 15, wherein the Type IIS restriction endonuclease is selected from the group consisting of BsgI, BpmI, BsmFl, MmeI and FokI.

18. The method of claim 15, wherein the reporter protein is a fluorescent protein.

19. The method of claim 18, wherein the fluorescent reporter protein is EGFP.

20. The method of claim 15, wherein the recombinase is Cre or FLP.

21. The method of claim 20, wherein the recombinase is fused to thymidine kinase or nitroreductase.
